(19) United States
(12) Patent Application Publication
(10) Pub. No.: US 2026/0334816 A1
(43) Pub. Date: Oct. 29, 2026

(54) FALL DETECTION DEVICE

(71) Applicant: TELECOM DESIGN, CANEJAN (FR)

(72) Inventors: Brice FASSI, PESSAC (FR); Stanislas MARLY, MERIGNAC (FR); Mehdi MOULINE, PESSAC (FR)

(21) Appl. No.: 19/542,910

(22) Filed: Feb. 18, 2026

(30) Foreign Application Priority Data
Apr. 24, 2025 (FR) 2504356

(51) Int. Cl. A61B 5/11; A61B 5/00

(52) U.S. Cl. CPC A61B 5/1117; A61B 5/7246; A61B 5/7264; A61B 5/746; A61B 2560/0257; A61B 2562/0219

(57) ABSTRACT

Device for detecting a fall of a person wearing the device. The device comprises a control unit, a memory, an accelerometer and an atmospheric pressure sensor. Each time it receives (102) N new acceleration measurements from the accelerometer, the control unit is configured to obtain (103) from the pressure sensor a new atmospheric pressure measurement and to record (104) the new acceleration measurements and the new atmospheric pressure measurement in the memory. An algorithm for detecting a shock profile representative of a fall is executed (105) from the acceleration measurements. In the event of detection (106) of a shock profile representative of a fall, the control unit is configured to execute (107) an algorithm for detecting a loss of altitude profile representative of a fall from the atmospheric pressure measurements and, in the event of detection (108), to trigger (109) an alert procedure.

### CROSS-REFERENCE TO RELATED APPLICATION

This application is based on and claims priority under 35 U.S.C. § 119 to French Patent Application No. 2504356, filed on Apr. 24, 2025, in the French National Intellectual Property Office, the disclosure of which is incorporated by reference herein in its entirety.

### BACKGROUND

### 1. Field

The present disclosure relates to a device for detecting the fall of a person wearing the device. This can be a bracelet, pendant, watch, or other type of electronic device intended to be worn by a person. In particular, the device can be connected to a monitoring device via a wireless communication system in order to trigger an alert when a fall is detected. The present disclosure also relates to a method for detecting a fall of a person by an electronic device. The present disclosure is particularly well suited for isolated persons experiencing a situation with a risk of falling (lone workers, elderly persons, athletes, etc.).

### 2. Prior Art

Automatic fall detection is a crucial issue in the area of assisting vulnerable people, especially elderly or isolated people. It aims to enable rapid intervention in the event of an incident, thereby reducing potential health consequences.

Many fall detection devices use an accelerometer, and are capable of detecting a fall from a sudden change in one or more components of the acceleration experienced by the body of the user. Although this approach is widely used, it has limitations in terms of reliability: impacts, rapid movements or unusual gestures can generate signals similar to those of a real fall, resulting in false positives.

Other systems use atmospheric pressure sensors, which estimate a change in altitude corresponding to a transition from standing to lying on the ground. However, pressure measurements are often sensitive to atmospheric variations and ambient conditions, which can affect detection accuracy. Additionally, these sensors typically need to operate continuously or with a high sampling rate to detect a fall in real time, resulting in significant power consumption, unsuitable for prolonged battery operation.

Solutions combining several sensors have been proposed to overcome these limitations. However, these devices are often complex to configure, and they generally do not take full advantage of the possible synergy between different sensor data, including acceleration and pressure.

Therefore, there is still a need for a fall detection device that has both high detection reliability and low energy consumption.

### SUMMARY

The objective of the present disclosure is to remedy all or some of the drawbacks of the prior art, especially those disclosed above.

For this purpose, and according to a first aspect, a device for the fall of a person wearing the device is proposed by the present disclosure. The device comprises a control unit, a memory, an accelerometer and a pressure sensor. The accelerometer is configured to provide linear acceleration measurements along three orthogonal axes, each acceleration measurement comprising a component along each of the three axes. The pressure sensor is configured to provide atmospheric pressure measurements. The control unit is configured to:
configure the accelerometer so that, when the accelerometer is in an active mode, it periodically provides the control unit with a number N of new acceleration measurements with a predetermined acquisition frequency,
when N new acceleration measurements are received from the accelerometer, obtain a new atmospheric pressure measurement from the pressure sensor and record the new acceleration measurements and the new atmospheric pressure measurement in the memory,
execute an algorithm for detecting a shock profile representative of a fall from acceleration measurements recorded in the memory,
if a shock profile representative of a fall is detected, execute a detection algorithm for an altitude loss profile representative of a fall from atmospheric pressure measurements recorded in the memory,
if a loss of altitude profile representative of a fall is detected, trigger an alert procedure.

In particular embodiments, the present disclosure may further comprise one or more of the following characteristics, considered individually or according to any technically possible combination.

In particular embodiments, the number N is between five and twenty.

In particular embodiments, the accelerometer is configured to switch autonomously from the active mode to a standby mode when several successive acceleration measurements are representative of a lack of movement of the person wearing the device, and to switch autonomously from the standby mode to the active mode when several successive acceleration measurements are representative of a movement of the person wearing the device.

In particular embodiments, following a switch of the accelerometer from the standby mode to the active mode, the control unit is configured to set the accelerometer with a first acquisition frequency during a wake-up period, then to set the accelerometer with a second acquisition frequency lower than the first acquisition frequency at the end of the wake-up period.

In particular embodiments, the first acquisition frequency is at least four times greater than the second acquisition frequency, or even at least eight times greater.

In particular embodiments, the control unit is configured to put the pressure sensor on standby between two new successive atmospheric pressure measurements. In particular embodiments, the control unit is configured to execute the algorithm for detecting an altitude loss profile after a predetermined time ΔT1, the algorithm being executed each time a new atmospheric pressure measurement is obtained until detection of an altitude loss profile or until expiration of a predetermined period ΔT2.

In particular embodiments, the parameters ΔT1 and ΔT2 are predetermined by a supervised machine learning algorithm trained from training elements each comprising acceleration measurements and atmospheric pressure measurements taken by the device during a particular scenario as well as an indication whether or not the scenario corresponds to a fall.

In particular embodiments, the algorithm for detecting an altitude loss profile representative of a fall comprises checking whether values determined from atmospheric pressure measurements recorded in the memory fall within a template. The template comprises chronologically a first zone corresponding to atmospheric pressure values varying little and associated with a high position of the device, a second zone corresponding to atmospheric pressure values varying quickly and associated with a loss of altitude of the device from the high position to a low position, and a third zone corresponding to atmospheric pressure measurements varying little and associated with the low position.

In particular embodiments, the first zone of the template is defined by a pressure range p1 and a duration t1, the second zone is defined by a pressure range p2 and a duration t2, the third zone is defined by a pressure range p3 and a duration t3. The set of parameters (t1, t2, t3, p1, p2, p3) is previously determined by a supervised machine learning algorithm trained from training elements each comprising atmospheric pressure measurements taken by the device during a particular scenario as well as an indication whether or not the scenario corresponds to a fall.

In particular embodiments, executing the algorithm for detecting a shock profile representative of a fall includes one or more of the following steps:
comparison with respect to a threshold of a difference between a long-term moving average and a short-term moving average of a norm of the acceleration measurements,
comparison with respect to a threshold of a norm of an acceleration measurement for several successive acceleration measurements,
comparison with respect to a threshold of a norm of a difference between two acceleration measurements close in time,
comparison with respect to a threshold of an angle between two vectors formed respectively by the three components of each of two acceleration measurements close in time.

In particular embodiments, the device comprises a wireless communication module, and the alert procedure comprises a transmission of an alert message to a monitoring device by the wireless communication module.

In particular embodiments, the alerting procedure comprises executing, by the control unit, an activity resumption detection algorithm according to at least one of the following criteria:
detecting, from atmospheric pressure measurements recorded in the memory after the triggering of the alert procedure, an altitude regain profile representative of a change from a lying position to a standing position of the person wearing the device,
detecting, from acceleration measurements recorded in the memory after the triggering of the alert procedure, movements representative of a resumption of activity of the person wearing the device.

In particular embodiments, the alert procedure comprises executing an algorithm for detecting a cancellation of the alert procedure, the cancellation being triggered via a user interface of the device.

According to a second aspect, the present disclosure relates to a method for detecting a fall of a person by a device. The device comprises a control unit, a memory, an accelerometer and a pressure sensor. The accelerometer is configured to provide linear acceleration measurements along three orthogonal axes, each acceleration measurement comprising a component along each of the three axes. The pressure sensor is configured to provide atmospheric pressure measurements. The method is implemented by the control unit and comprises:
configuring the accelerometer so that, when the accelerometer is in an active mode, it periodically provides the control unit with a predetermined number N of new acceleration measurements with a predetermined acquisition frequency,
when N new acceleration measurements are received from the accelerometer:
   obtaining a new atmospheric pressure measurement from the pressure sensor,
   recording the new acceleration measurements and the new atmospheric pressure measurement in the memory,
executing an algorithm for detecting a shock profile representative of a fall from acceleration measurements recorded in the memory,
in the event of detection of a shock profile representative of a fall, executing an algorithm for detecting an altitude loss profile representative of a fall from atmospheric pressure measurements recorded in the memory,
in the event of detection of an altitude loss profile representative of a fall, triggering an alert procedure.

According to a third aspect, the present disclosure relates to a computer-readable non-volatile storage medium comprising a set of program code instructions intended to configure a control unit of a fall detection device. The device comprises a control unit, a memory, an accelerometer and a pressure sensor. The accelerometer is configured to provide linear acceleration measurements along three orthogonal axes, each acceleration measurement comprising a component along each of the three axes. The pressure sensor is configured to provide atmospheric pressure measurements. The program code instructions are intended to configure the control unit to implement a method according to any one of the implementations of the detection method according to the present disclosure.

### BRIEF DESCRIPTION OF THE DRAWINGS

The present disclosure will be better understood upon reading the following description, given by way of non-limiting example, and made with reference to the following figures:

FIG. 1 an example embodiment of a fall detection device according to the present disclosure,

FIG. 2 the main steps of a fall detection method according to the present disclosure,

FIG. 3 an example of implementing an alert procedure following the detection of a fall,

FIG. 4 an example of implementing an algorithm for detecting a shock profile representative of a fall,

FIG. 5 an example of implementing an algorithm for detecting an altitude loss profile representative of a fall,

FIG. 6 a chronological illustration of an execution of the algorithm for detecting a shock profile followed by an execution of the algorithm for detecting an altitude loss profile.

In these figures, identical references from one figure to another refer to identical or similar elements. For clarity, the elements shown are not necessarily plotted to the same scale, unless stated otherwise.

### DETAILED DESCRIPTION

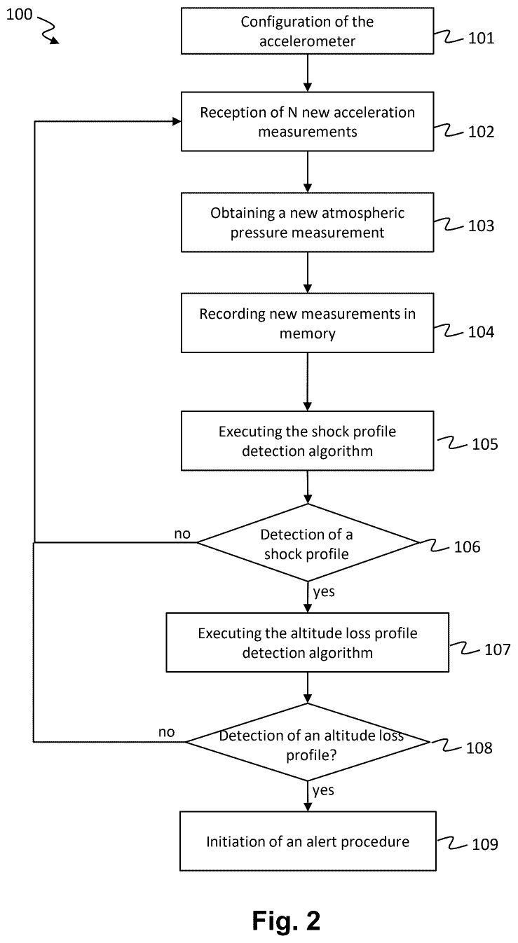

FIG. 2 schematically shows a fall detection device 10 according to the present disclosure. The device 10 is intended to be worn by a person in order to automatically detect a fall event and to initiate, if necessary, an alert procedure.

This may in particular be a bracelet, a pendant, a watch, or another type of electronic device intended to be worn by a person, for example on a belt, on a cuff, or even in a pocket of a garment. According to another example, the device 10 could be integrated in a textile of a garment. However, these examples are in no way limiting, and nothing would prevent the device 10 from taking the form of another accessory or housing worn by a person.

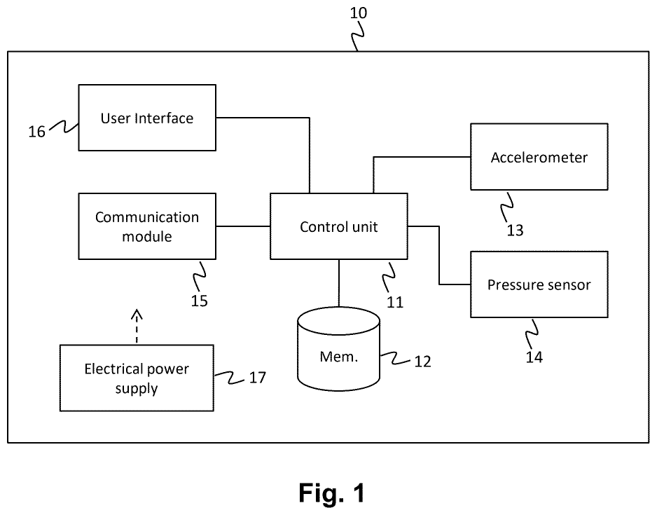

As illustrated in FIG. 1, the device 10 comprises a control unit 11, a memory 12, an accelerometer 13, a pressure sensor 14, and an electrical energy supply module 17. The control unit 11 is connected to the memory 12, to the accelerometer 13 and to the pressure sensor 14. In particular, the control unit 11 is configured to record the measurements received from the sensors 13 and 14 in the memory 12, to process these measurements by executing a fall detection algorithm and, if applicable, to trigger an alert procedure.

The control unit 11 may also comprise a wireless communication module 15 and a user interface 16 which, where applicable, are also connected to the control unit 11. In particular, the wireless communication module 15 can make it possible to send an alert message to a monitoring device when a fall is detected. The monitoring device may correspond for example to a remote monitoring server, or to the cell phone of a person responsible for monitoring the person wearing the device 10.

The wireless communication module 15 can, for example, implement the Bluetooth or BLE (“Bluetooth Low Energy”) communication standard to communicate with a cell phone or a telecare base located at a near distance. According to another example, the wireless communication module 15 may implement a cellular communication standard (3G, 4G or 5G for example) or a communication protocol of the LPWAN type (the acronym for “Low Power Wide Area Network”) to communicate with a remote monitoring server or a remote cell phone. The communication module 15 comprises, for example, a radio circuit for transmitting and receiving wireless signals, an antenna adapted to the frequency band used, and a communication controller (or transceiver) responsible for managing the transmission protocols.

The electrical energy supply module 17 is intended to provide the energy necessary for the operation of the various components of the device 10, in particular the control unit 11, the accelerometer 13, the pressure sensor 14, the memory 12, the communication module 15 and the user interface 16. The power supply module 17 may have different forms depending on the constraints of integration, battery life and use of the device. This may include a rechargeable battery, such as a lithium-ion battery, or replaceable batteries. It could also be an energy harvesting circuit capable of capturing energy from the environment and converting it into electrical energy usable by the device 10.

The accelerometer 13 is configured to provide acceleration measurements along three orthogonal axes. This can be an integrated three-axis accelerometer, or a combination of three single-axis accelerometers, each oriented along one of the three axes. In general, the acceleration sensor may take any form known in the prior art, provided that it makes it possible to provide linear acceleration measurements along three orthogonal axes, each acceleration measurement comprising a component along each of the three axes.

The pressure sensor 14 is adapted to provide measurements representative of the ambient air pressure. These measurements are then used by the control unit 11 to estimate relative altitude variations. This can be a MEMS (for “Micro-Electro-Mechanical System”) barometric sensor or a capacitive barometric sensor. The pressure sensor may be combined with software and/or hardware filtering to smooth natural variations in atmospheric pressure due to environmental changes, slow movements or weather conditions, and thus better detect sudden changes corresponding to a fall. Advantageously, the pressure sensor 14 can be put on standby each time a new pressure measurement is obtained by the control unit 11, in order to reduce the electrical energy consumption of the pressure sensor 14 and therefore also of the device 10.

The various components of the fall detection device 10 may be integrated in a single housing, for example in a watch, a pendant, or another connected accessory. However, nothing would prevent, in a variant, various components of the device 10 from being integrated in separate physical entities. For example, it could be imagined that the control unit 11 and/or the wireless communication module 15 are located within a first physical entity, while the accelerometer 13 and the pressure sensor 14 are located in a second physical entity distinct from the first. For example, the control unit 11 and/or the wireless communication module 15 could be integrated in a mobile phone, while the sensors 13 and 14 would be integrated in a dedicated accessory or housing worn elsewhere on the body. In such a variant, the physical entity comprising the sensors 13 and 14 would be connected to the control unit 11 by a wired or wireless communication link.

The main function of the user interface 16 is to enable the person wearing the device 10 to interact with it in a simple and intuitive manner. It provides an input channel for the user to transmit an intent (e.g. to cancel an alert or manually trigger assistance) and/or an output channel for providing feedback (e.g. to confirm an action or indicate an event visually, acoustically or tactilely). In particular, the user interface 16 can be used to indicate to the user the detection of a fall. For this purpose, the user interface 16 may comprise a light indicator such as an LED, an audible or touch feedback device (speaker, vibrator), or a display screen. The user interface 16 may also allow the user to confirm or negate the detection of a fall. It may be, for example, a physical button or a capacitive touch sensor positioned on the housing of the device 10, or a virtual interface implemented, for example, by means of a touch screen of the device 10.

The control unit 11 is responsible for coordinating the entire operation of the device 10, in particular controlling the sensors 13 and 14, processing the measurements, executing the detection algorithms, managing the memory 12, controlling the communication module 15 and controlling the user interface 16. It can be implemented in various forms depending on performance, consumption, size or cost requirements. Examples include an integrated microcontroller (MCU for “Micro-Controller Unit”), a digital signal processor (DSP), a system-on-chip (SoC), a programmable logic device (PLD) or FPGA (for “Field-Programmable Gate Array”), etc.

The control unit 11 may be configured by a set of program code instructions stored in a non-volatile memory to implement a fall detection method.

FIG. 2 schematically represents the main steps of a fall detection method 100 implemented by the control unit 11 of the fall detection device 10.

Firstly, the control unit 11 implements a step of configuring 101 the accelerometer 13 so that, when it is in an active mode, it periodically provides the control unit 11 with a number N of new acceleration measurements with a predetermined acquisition frequency. P is a natural integer greater than or equal to one.

As illustrated in FIG. 2, each time the control unit 11 receives N new acceleration measurements from the accelerometer 13, it obtains a new atmospheric pressure measurement from the pressure sensor 14 (step 103). Then, the control unit 11 stores the new acceleration measurements and the new atmospheric pressure measurement in the memory 12 (step 104). An algorithm for detecting a shock profile is then executed (step 105) from the acceleration measurements recorded in the memory. If a shock profile representative of a fall is detected (step 106), a second algorithm analyzes the atmospheric pressure measurements to detect an altitude loss profile typical of a fall (step 107). If an altitude loss profile representative of a fall is detected (step 108), then an alert procedure is triggered (step 109).

The algorithm for detecting a shock profile can be executed each time the control unit 11 receives N new acceleration measurements from the accelerometer 13. However, it is not essential that the algorithm for detecting a shock profile be executed synchronously with the reception of N new acceleration measurements of the accelerometer 13. For example, it could be executed more frequently or less frequently.

The choice of the value N results from a compromise between energy consumption and detection reliability. A high number N makes it possible to reduce the frequency at which the pressure measurements must be acquired, which decreases the stress on the pressure sensor 14, generally more energy-consuming, and thus improves the overall battery life of the device 10. Conversely, a lower number N makes it possible to obtain a higher frequency of atmospheric pressure measurements, which can make it possible to identify a drop more reliably, but at the cost of increased consumption. In particular, the value N can be selected between five and twenty (however, nothing would prevent other values from being considered).

The accelerometer 13 can operate in an active mode or in a standby mode. Unlike active mode, standby mode corresponds to a mode in which the functionalities of the accelerometer 13 are reduced in order to limit its energy consumption. The accelerometer 13 can be configured by the control unit 11 to switch autonomously to standby mode when several successive acceleration measurements have values representative of a significant absence of movement (for example with a relatively low and stable acceleration amplitude) and to return to active mode as soon as a movement is detected, for example when one or more successive measurements show an amplitude above a certain threshold.

Following a switch of the accelerometer 13 from the standby mode to the active mode, the control unit 11 can be configured to set the accelerometer 13 with a first acquisition frequency during a wake-up period, then to set the accelerometer 13 with a second acquisition frequency lower than the first acquisition frequency at the end of the wake-up period. The wake-up period, with a higher acquisition frequency, makes it possible to best capture the initial dynamics of the movement after a sleep period.

Switching back to a lower acquisition frequency after a certain time makes it possible to optimize the battery life of the device 10. Typically, the acquisition frequency during a wake-up period may be four to eight times higher than during normal operation (active mode excluding wake-up period). This mechanism of dynamic adaptation of the acquisition frequency of the acceleration measurements optimizes the detection performance while preserving the battery life of the device 10. The wake-up period can typically last a few seconds.

By way of non-limiting example, the accelerometer 13 may be configured to periodically provide twelve (N=12) new acceleration measurements to the control unit 11 when it is in active mode. In normal operation (outside the wake-up period), the accelerometer 13 can be configured with an acquisition frequency of 25 Hz (acquisition of twenty-five acceleration measurements per second by the accelerometer 13). In this case, the controller obtains twelve new acceleration measurements and one new atmospheric pressure measurement every 480 ms). During the wake-up period, the accelerometer 13 can be configured with an acquisition frequency of 200 Hz (acquisition of two hundred acceleration measurements per second by the accelerometer 13), which leads to obtaining twelve new acceleration measurements and a new atmospheric pressure measurement every 60 ms by the control unit.

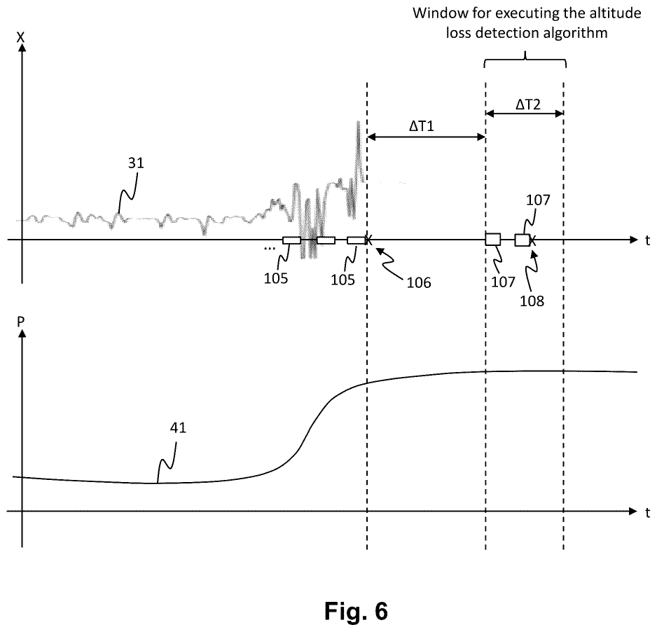

Obtaining 103 a new atmospheric pressure measurement from the pressure sensor 14 is demanded by the control unit 11 each time a set of N new acceleration measurements is obtained from the accelerometer 13. This advantageously makes it possible to synchronize the atmospheric pressure measurements with the acceleration measurements (unlike a scenario where the pressure sensor 14 and the accelerometer 13 would each provide measurements to the control unit 11 asynchronously). As will be explained in more detail later with reference to FIG. 6, this allows for better synchronization between the execution of the algorithm for detecting a shock profile and the execution of the algorithm for detecting an altitude loss profile, as well as better fall detection reliability (reduction in the number of false detections). This can also make it possible to optimize the battery life of the fall detection device 10 by putting the pressure sensor 14 on standby between two atmospheric pressure measurements.

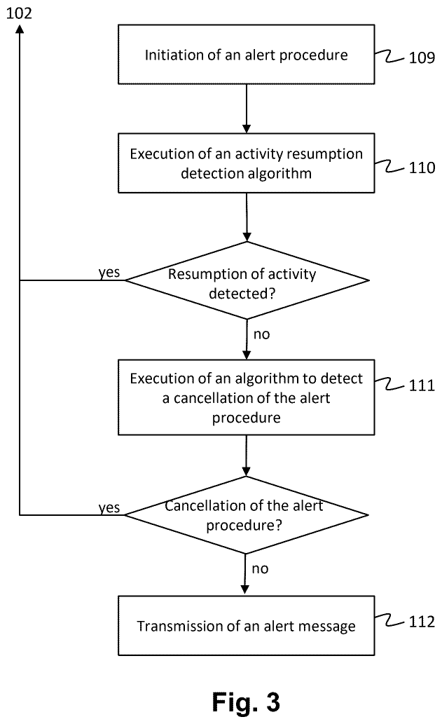

FIG. 3 shows an example of implementation of an alert procedure by the device 10 when the two detection algorithms are positive, i.e. when a shock profile and a loss of altitude profile typical of a fall are successively detected.

In the example illustrated in FIG. 3, the alert procedure comprises an execution of an algorithm for detecting resumption of activity by the person wearing the device 10 (step 110). It is for example possible to detect an altitude regain profile representative of a change from a lying position to a standing position of the person wearing the device 10. This detection of an altitude regain profile may be executed from atmospheric pressure measurements recorded in the memory 12 after the triggering 109 of the alert procedure. According to another example, it can be envisaged detecting movements representative of a resumption of activity of the person from acceleration measurements recorded in the memory 12 after the triggering 109 of the alert procedure (for example stepping movements). However, nothing would prevent envisaging other criteria for the detection of a resumption of activity, or a combination of these criteria.

As illustrated in FIG. 3, if a resumption of activity is detected, the fall detection device 10 resumes its normal operation (return to step 102). If no resumption of activity is detected, the alert procedure includes an execution of an algorithm for detecting a cancellation of the alert procedure (step 111). For example, an indication of fall detection can be given to the person wearing the device 10 via the user interface 16, for example via an audible indication, a light indication, a vibration, or a text message. If the person wearing the device 10 deems that they do not need assistance, they can cancel the alert procedure, always via the user interface 16, for example by pressing a physical button, placing a finger on a capacitive sensor, or pressing a virtual button of a graphical interface displayed on a screen.

As illustrated in FIG. 3, if the alert procedure is canceled, the fall detection device 10 resumes its normal operation (return to step 102). On the other hand, if no cancellation of the alert procedure is detected for a predetermined time, then an alert message is issued (step 112). In particular, this may be a radio message transmitted by the wireless communication module 15 to a monitoring device (e.g. a remote monitoring server, the cell phone of a person responsible for monitoring the person carrying the device 10, or another remote care device). In a complementary manner or alternatively, the alert message may take the form of an audible signal making it possible to alert a person relatively close to the person who is the victim of the fall (the communication module 15 may therefore be optional).

The alert procedure shown in FIG. 3 is only an example. In particular, the steps of detecting a resumption of activity (step 110) and detecting a cancellation of the alert procedure (step 111) are optional (in which case the user interface 16 is not essential).

The algorithm for detecting a shock profile (in step 105) may be implemented according to various variants, relying on the acceleration measurements obtained from the accelerometer 13 and recorded in the memory 12.

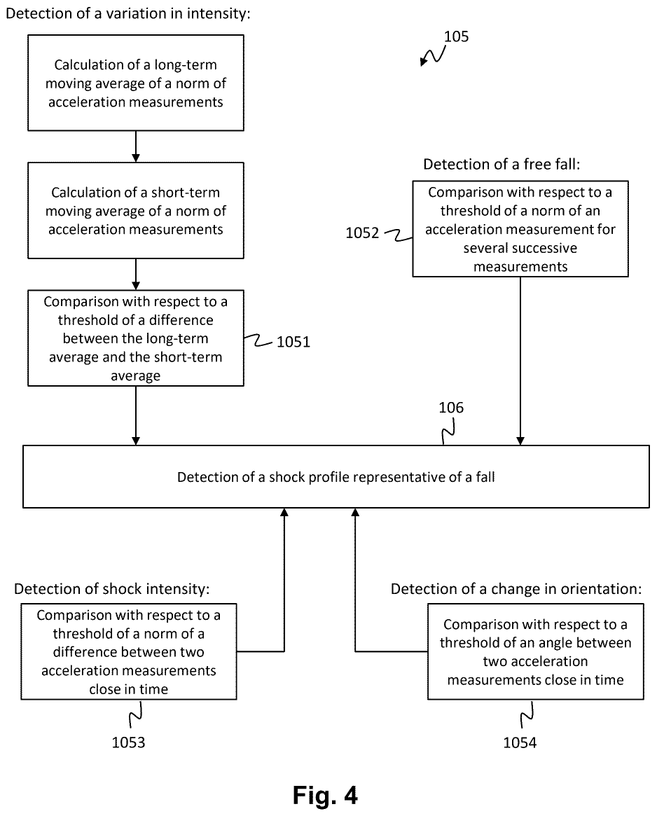

FIG. 4 illustrates an example of implementation of the algorithm for detecting a shock profile typical of a fall. In this example, the algorithm considers various criteria, in particular the detection of a variation in intensity of the movements, the detection of a free fall, the detection of a high shock intensity, and the detection of a change in orientation of the movements. In variants, it would be conceivable to consider only a subset of these criteria or to consider other criteria.

The detection of a variation in the intensity of movements corresponds to the detection of a rapid increase in the average intensity of movements (this increase being characteristic of a fall). As illustrated in FIG. 4, to evaluate this criterion, a long-term moving average and a short-term moving average of a norm of the acceleration measurements recorded in the memory 12 are calculated. For example, the norm of an acceleration measurement corresponds to the sum of the squares of the three components of the measurement along the three orthogonal axes (N²=X²+Y²+Z²). For example, the long-term average is calculated on the last sixty-four measurements obtained from the accelerometer 13, while the short-term average is calculated on the last four measurements (however, other values could be envisaged for the number of measurements to be considered for calculating the long-term average and the short-term average). A difference (for example the difference in absolute value) between the long-term average and the short-term average can then be compared with a threshold (step 1051). If this difference is greater than the threshold, this means that there was a rapid increase in the average intensity of movements (this increase being characteristic of a fall).

The detection of a free fall may correspond to the detection of a cancellation of gravity, for example when the norm of several successive acceleration measurements is less than a threshold (step 1052). A free fall period observed by the accelerometer 13 is also typical of a fall of the person wearing the device 10.

The detection of a high shock intensity may comprise the comparison with respect to a threshold of a norm of a difference between two acceleration measurements close in time (step 1053). The norm of the difference between two acceleration measurements corresponds, for example, to the sum of the squares of the differences calculated for each component (N²(Δ)=ΔX²+ΔY²+Δz²). This is the norm of the vector difference between the two measurements. The two acceleration measurements that are close in time may, for example, correspond to two successive measurements, or to two measurements separated by a few intermediate measurements (for example less than a dozen intermediate measurements).

The detection of a sudden change in orientation of two temporally close acceleration measurements may correspond to the detection of a rebound of the accelerometer weight, which is characteristic of an end of fall. As illustrated in FIG. 4, this detection may comprise comparing with respect to a threshold an angle between two vectors formed respectively by the three components of each of two acceleration measurements close in time (step 1054).

These various criteria can be combined in a hybrid algorithm, for example using a logical rule system, a decision tree or a classifier learned by machine learning.

In an even more advanced variant, a convolutional or recurrent neural network can be trained from time series of acceleration measurements to automatically identify characteristic fall patterns, including in complex cases where simple thresholds are not sufficient.

The algorithm for detecting an altitude loss profile (in step 107) may be implemented according to various variants, relying on the atmospheric pressure measurements obtained from the pressure sensor 14 and recorded in the memory 12.

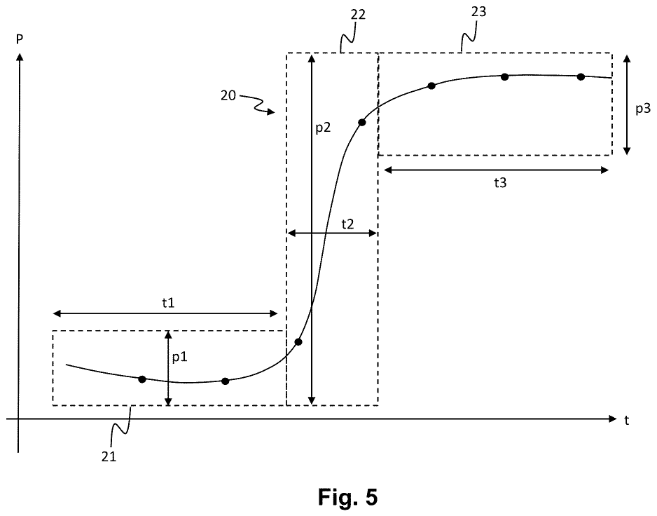

FIG. 5 schematically represents an example of implementation of the algorithm for detecting an altitude loss profile typical of a fall. In this example, the algorithm relies on the detection of a predefined template 20 of variation in the atmospheric pressure over time. Typically, this template 20 comprises chronologically a first zone 21 corresponding to atmospheric pressure values varying little and associated with a high position of the device 10 (position corresponding to a standing position of the person wearing the device), a second zone 22 corresponding to rapidly varying atmospheric pressure values and associated with a loss of altitude of the device 10 from the high position to a low position (the low position corresponds to a position lying on the ground of the person following a fall), and a third zone 23 corresponding to atmospheric pressure measurements varying little and associated with the low position. The first zone 21 is defined by a pressure range p1 and a duration t1; the second zone 22 is defined by a pressure range p2 and a duration t2; the third zone 23 is defined by a pressure range p3 and a duration t3. The durations t1 and t2 are significantly longer than the duration t3. The pressure ranges p1 and p2 are significantly lower than the pressure range p3. An increase in atmospheric pressure measurements corresponds to a loss of altitude.

In the example illustrated in FIG. 5, the algorithm consists in checking whether values determined from recent atmospheric pressure measurements recorded in the memory 12 fall within the template 20. The values compared with the template may correspond directly to the atmospheric pressure measurements recorded in the memory, or to a subset of these measurements, or they may be derived from a filtering of these measurements, or correspond to points on a curve approximately followed by these measurements.

The set of parameters (t1, t2, t3, p1, p2, p3) may be determined in advance empirically from test campaigns, or by a supervised machine learning algorithm trained from fall and non-fall scenario training elements. Each training element comprises atmospheric pressure measurements taken by the device 10 during a particular scenario as well as an indication whether or not the scenario corresponds to a fall.

In a variant, the algorithm for detecting an altitude loss profile may be based on an analysis of the pressure measurements by a statistical method or by Bayesian modeling making it possible to assess the probability that a sequence belongs to a “fall” class or a “non-fall” class according to its time profile.

According to yet another variant, the algorithm may consist in encoding the pressure measurements in the form of characteristic vectors on sliding time windows, which would then be analyzed by a supervised machine learning model (SVM, decision tree or random forest model).

In an even more advanced variant, detection of the deep learning type can be implemented with a recurrent neural network trained to recognize typical fall sequences in pressure signals. In particular, these methods enable adaptation to varying environmental conditions or different user profiles and improve robustness against artifacts and atypical movements.

FIG. 6 is a chronological illustration of an execution 105 of the algorithm for detecting a shock profile followed by an execution 107 of the algorithm for detecting an altitude loss profile.

In FIG. 6, curve 31 represents the change over time in a component (for example along the X axis) of the acceleration measurements, while curve 41 represents the change over time in the atmospheric pressure measurements. Only one component of the acceleration measurements is shown in FIG. 6 for the sake of clarity. However, it should be noted that the algorithm for detecting a shock profile takes into account the three components of acceleration measurements.

As illustrated in FIG. 6, the algorithm for detecting a shock profile representative of a fall is executed recurrently, for example each time new acceleration measurements are received from the accelerometer 13. Detection 106 of a shock profile representative of a fall causes execution 107 of the algorithm for detecting an altitude loss profile in a time window. The time window starts after a time ΔT1 after the detection 106 of a shock profile representative of a fall. The time window has a duration ΔT2. The algorithm for detecting an altitude loss profile can be executed several times in the time window, for example each time a new pressure measurement is obtained. If an altitude loss profile representative of a fall is detected in the time window, then an alert procedure is triggered. On the other hand, if no altitude loss profile representative of a fall is detected in the time window, then the device 10 resumes its normal operation.

The parameters ΔT1 and ΔT2 play an important role in the reliability of fall detection. The duration ΔT1 defines the interval between the detection 106 of a shock profile representative of a fall and the start of the time window in which the algorithm for detecting an altitude loss profile is executed one or more times. The duration ΔT2 corresponds to the duration of the time window in which the algorithm for detecting an altitude loss profile is executed to confirm/negate the detection of a typical shock profile of a fall. The parameters ΔT1 and ΔT2 strongly depend on the technical and structural characteristics of the device 10, and in particular on the pressure sensor 14 and its seal, but also on the characteristics of the housing of the device 10 (material, shape, rigidity, etc.). The parameters ΔT1 and ΔT2 must be determined accurately to ensure good reliability of the fall detection, thus avoiding false detections (false positives). If it is suitably selected, the parameter ΔT2 makes it possible to optimize the electrical consumption of the device 10.

The parameters ΔT1 and ΔT2 can be determined in advance empirically. They may also be determined by a supervised machine learning algorithm trained from training elements each comprising acceleration measurements and atmospheric pressure measurements taken by the device 10 during a particular scenario as well as an indication whether or not the scenario corresponds to a fall.

The above description clearly illustrates that, by its different features and their advantages, the present present disclosure achieves the objectives set, namely offering a solution for detecting a fall of an isolated person having both high detection reliability and low electrical energy consumption.

More generally, it should be noted that the implementations and embodiments considered hereinabove have been described as non-limiting examples, and that other variants could consequently be considered.

In particular, the example described with reference to FIG. 4 for the implementation of the algorithm for detecting a shock profile typical of a fall and the example described with reference to FIG. 5 for the algorithm for detecting an altitude loss profile typical of a fall are not limiting. Other implementations could be envisaged. The core of the present disclosure relies in particular on a combined use of acceleration measurements and atmospheric pressure measurements, with a particular chronology of the detection of a shock profile followed by the detection of an altitude loss profile.

What is claimed is:

1. A device for detecting the fall of a person wearing the device, the device comprising:
a control unit,
a memory,
an accelerometer configured to provide linear acceleration measurements along three orthogonal axes, each acceleration measurement comprising a component along each of the three axes,
a pressure sensor configured to provide atmospheric pressure measurements,
the control unit being configured to:
configure the accelerometer so that, when the accelerometer is in an active mode, it periodically provides the control unit with a number N of new acceleration measurements with a predetermined acquisition frequency,
when N new acceleration measurements are received from the accelerometer, obtain from the pressure sensor a new atmospheric pressure measurement and record the new acceleration measurements and the new atmospheric pressure measurement in the memory,
execute an algorithm for detecting a shock profile representative of a fall from acceleration measurements recorded in the memory,
in the event of detection of a shock profile representative of a fall, execute an algorithm for detecting an altitude loss profile representative of a fall from atmospheric pressure measurements recorded in the memory,
in the event of detection of an altitude loss profile representative of a fall, trigger an alert procedure.

2. The device according to claim 1, wherein N is between five and twenty.

3. The device according to claim 1, wherein the accelerometer is configured to switch autonomously from the active mode to a standby mode when several successive acceleration measurements are representative of a lack of movement of the person wearing the device, and to switch autonomously from the standby mode to the active mode when several successive acceleration measurements are representative of a movement of the person carrying the device.

4. The device according to claim 3, wherein, following a switch of the accelerometer from the standby mode to the active mode, the control unit is configured to set the accelerometer with a first acquisition frequency during a wake-up period, then to set the accelerometer with a second acquisition frequency lower than the first acquisition frequency at the end of the wake-up period.

5. The device according to claim 4, wherein the first acquisition frequency is at least four times higher than the second acquisition frequency, or even at least eight times higher.

6. The device according to claim 1, wherein the control unit is configured to put the pressure sensor on standby between two new successive atmospheric pressure measurements.

7. The device according to claim 1, wherein the control unit is configured to execute the algorithm for detecting an altitude loss profile after a predetermined time ΔT1, the algorithm being executed each time a new atmospheric pressure measurement is obtained until an altitude loss profile is detected or until a predetermined period ΔT2 has expired.

8. The device according to claim 7, wherein the parameters ΔT1 and ΔT2 are predetermined in advance by a supervised machine learning algorithm trained from training elements each comprising acceleration measurements and atmospheric pressure measurements taken by the device during a particular scenario, as well as an indication whether the scenario corresponds to a fall or not.

9. The device according to claim 1, wherein the algorithm for detecting an altitude loss profile representative of a fall comprises a check whether values determined from atmospheric pressure measurements recorded in the memory fall within a template, the template chronologically comprising a first zone corresponding to atmospheric pressure values varying little and associated with a high position of the device, a second zone corresponding to atmospheric pressure values varying quickly and associated with an altitude loss of the device from the high position to a low position, and a third zone corresponding to atmospheric pressure measurements varying little and associated with the low position.

10. The device according to claim 9, wherein the first zone of the template is defined by a pressure range p1 and a duration t1, the second zone is defined by a pressure range p2 and a duration t2, the third zone is defined by a pressure range p3 and a duration t3, and the set of parameters (t1, t2, t3, p1, p2, p3) is previously determined by a supervised machine learning algorithm trained from training elements each comprising atmospheric pressure measurements taken by the device during a particular scenario as well as an indication whether or not the scenario corresponds to a fall.

11. The device according to claim 1, wherein the execution of the algorithm for detecting a shock profile representative of a fall comprises one or more of the following steps:
comparing with respect to a threshold a difference between a long-term moving average and a short-term moving average of a standard of the acceleration measurements,
comparing with respect to a threshold a norm of an acceleration measurement for several successive acceleration measurements,
comparing with respect to a threshold a norm of a difference between two acceleration measurements close in time,
comparing with respect to a threshold an angle between two vectors formed respectively by the three components of each of two acceleration measurements close in time.

12. The device according to claim 1, wherein the device comprises a wireless communication module, and the alert procedure comprises transmitting an alert message to a monitoring device by the wireless communication module.

13. The device according to claim 1, wherein the alert procedure comprises an execution, by the control unit, of an activity resumption detection algorithm according to at least one of the following criteria:
detecting, from atmospheric pressure measurements recorded in the memory after the triggering of the alert procedure, an altitude regain profile representative of a change from a lying position to a standing position of the person wearing the device,
detecting, from acceleration measurements recorded in the memory after the triggering of the alert procedure, movements representative of a resumption of activity of the person wearing the device.

14. The device according to claim 1, wherein the alert procedure comprises an execution of an algorithm for detecting a cancellation of the alert procedure, the cancellation being triggered via a user interface of the device.

15. A method for detecting a fall of a person by a device comprising:
a control unit,
a memory,
an accelerometer configured to provide linear acceleration measurements along three orthogonal axes, each acceleration measurement comprising a component along each of the three axes,
a pressure sensor configured to provide atmospheric pressure measurements,
the method being implemented by the control unit and comprising:
configuring the accelerometer so that, when the accelerometer is in an active mode, it periodically provides the control unit with a predetermined number N of new acceleration measurements with a predetermined acquisition frequency,
when N new acceleration measurements are received from the accelerometer:
obtaining a new atmospheric pressure measurement from the pressure sensor,
recording the new acceleration measurements and the new atmospheric pressure measurement in the memory,
executing an algorithm for detecting a shock profile representative of a fall from acceleration measurements recorded in the memory,
in the event of detection of a shock profile representative of a fall, executing an algorithm for detecting a loss of altitude profile representative of a fall from atmospheric pressure measurements recorded in the memory,
in the event of detection of an altitude loss profile representative of a fall, triggering an alert procedure.

16. A computer-readable non-volatile storage medium comprising a set of program code instructions intended to configure a control unit of a fall detection device, the program code instructions being intended to configure the control unit to implement a method according to claim 15.
